(19) United States
(12) Patent Application Publication
(10) Pub. No.: US 2009/0022810 A1
(43) Pub. Date: Jan. 22, 2009

(54) EmunoTM Bar, by Nutra-ShieldTM

(76) Inventor: Rudolph Silas, Glen Clove, NY (US)

Correspondence Address: NHANCE, LLC, 175 REMSEN STREET, SUITE 602, BROOKLYN, NY, 11201, US

(21) Appl. No.: 11/879,706

(22) Filed: Jul. 19, 2007

(51) Int. Cl. A61K 35/20

(52) U.S. Cl. 424/535

(57) ABSTRACT

The invention, Emuno Bar by NutraShield, is a nutraceutical food bar composed of all natural ingredients that are rich in antioxidants and fortified with the patented Formulation 100 (U.S. Pat. No. 6,667,063, B2). The invention is a dedicated delivery system for U.S. Pat. No. 6,667,063, B2 and designed to boost the human immune system. The Emuno Bar will be the only Dark Chocolate covered functional food bar containing the new revolutionary patented Formulation 100.

### BRIEF DESCRIPTION OF THE SEVERAL VIEWS OF THE DRAWING

Not Applicable

### DESCRIPTION

The Emuno™ Bar, by NutraShield™ is a dedicated delivery system for Immune Formulation 100™ (U.S. Pat. No. 6,667,063 B2) and dietary nutraceutical food bar. The Emuno™ Bar is rich in antioxidants and, unlike other health bars, it is primarily designed to enhance (boost) the human immune system.

The Emuno™ Bars is composed of the following ingredients:

| | | | |
|---|---|---|---|
| (1) | Dried Raspberries | 5 | grams |
| (2) | Dried Blueberries | 2.5 | grams |
| (3) | Sweetened Coconut Flakes | 2.5 | grams |
| (4) | Diced Raw Almonds | 5 | grams |
| (5) | Puffed Rice | 1.25 | grams |
| (6) | Honey | 10 | grams |
| (7) | Patented Formulation 100 ™ | 1 | gram |
| | (U.S. Pat. No. 6,667,063 B2) | | |
| (8) | Dark Chocolate: (minimum 68% Cocoa), | 12.75 | grams |
| | (sugar, chocolate liquor, cocoa butter, | | |
| | anhydrous milk fat, soy, and lecithin) | | |
| | Total | 40 | gram bar |

Nutritional Profile

The method of preparing the Emuno™ Bar is as follows:
(1) Combine all dry ingredients, except for Patented Formulation # U.S. Pat. No. 6,667,063 B2.
(2) Mix dry ingredients until well blended and evenly distributed.
(3) Cook Honey to minimum of 244° F., (118° C.).
(4) Pour cooked, hot Honey onto dry ingredients, and mix to distribute Honey evenly.
(5) Cool to at least 120° F., (50° C.) before adding Patented Formulation to avoid degrading immune proteins.
(6) After cooling, sift in Patented Formulation # U.S. Pat. No. 6,667,063 B2 and mix to distribute evenly.
(7) Spread onto oiled parchment paper and roll to approximately ¼ inch thick by approximately 3 inches in length by 1.5 inches in. width.
(8) Enrobe bar with tempered Dark Chocolate, thinned with approximately 15% Cocoa Butter.

DEFINITION LIST 1

| TERM | DEFINITION |
|---|---|
| Nutraceutical | Any substance that is a food or a part of a |
| | food and provides medical or health |
| | benefits, including the prevention and |
| | treatment of disease. Such products may |
| | range from isolated nutrients, dietary |
| | supplements and specific diets to |
| | genetically engineered designer foods, |
| | herbal products and processed foods, such |
| | as cereals, soups and beverages. It is |
| | important to note that this definition |
| | applies to all categories of food and parts of |
| | food, ranging from dietary supplements, |
| | such as folic acid - used for the prevention |
| | of Spina Bifida - to chicken soup - taken to |
| | lessen the discomfort of the common cold. |
| | This definition also includes a bio- |
| | engineered designer vegetable food, rich in |
| | antioxidant ingredients, and a stimulant |
| | functional food or pharmafood. |

1. The claim for this application is the dedicated delivery system and dietary nutraceutical food bar of the Emuno™ Bar, by NutraShield™, which is as follows:

| INGREDIENTS | | AMOUNTS | |
|---|---|---|---|
| (1) | Dried Raspberries | 5 | grams |
| (2) | Dried Blueberries | 2.5 | grams |
| (3) | Sweetened Coconut Flakes | 2.5 | grams |
| (5) | Diced Raw Almonds | 5 | grams |
| (5) | Puffed Rice | 1.25 | grams |
| (9) | Honey | 10 | grams |
| (10) | Patented Formulation 100 ™ | 1 | gram |
| | (U.S. Pat. No. 6,667,063 B2) | | |
| (11) | Dark Chocolate: (minimum 68% Cocoa), | 12.75 | grams |
| | (sugar, chocolate liquor, cocoa butter, | | |
| | anhydrous milk fat, soy, and lecithin) | | |
| | Total | 40 | gram bar |

Nutritional Profile

### Method:

1. Combine all dry ingredients, except for Patented Formulation # U.S. Pat. No. 6,667,063 B2.
2. Mix dry ingredients until well blended and evenly distributed.
3. Cook Honey to minimum of 244° F., (118° C.).
4. Pour cooked, hot Honey onto dry ingredients, and mix to distribute Honey evenly.
5. Cool to at least 120° F., (50° C.) before adding Patented Formulation to avoid degrading immune proteins.
6. After cooling, sift in Patented Formulation # U.S. Pat. No. 6,667,063 B2 and mix to distribute evenly.
7. Spread onto oiled parchment paper and roll to approximately ¼ inch thick by approximately 3 inches in length by 1.5 inches in. width.
8. Enrobe bar with tempered Dark Chocolate, thinned with approximately 15% Cocoa Butter.
The Emuno™ Bar product is a dedicated delivery system and dietary nutraceutical food bar composed of the patented Immune Formulation 100™ and other natural ingredients rich in antioxidants. The patented Formulation is intended to enhance the immune system by raising the glutathione level in the human body.

DEFINITION LIST 2

| TERM | DEFINITION |
|---|---|
| Glutathione | A family of enzymes that utilize |
| | glutathione in reactions contributing to |
| | the transformation of a wide range of |
| | compounds, including carcinogens, |
| | therapeutic drugs and products of |
| | oxidative stress. These enzymes play a |
| | key role in the detoxification of such |
| | substances. |

What I claim as my invention is:

1. A dietary nutraceutical food bar composed of, Dried Raspberries, Dried Blueberries, Sweetened Coconut, Raw Almonds, Puffed Rice, Honey, Dark Chocolate, Immune Formulation 100 (U.S. Pat. No. 6,667,063, B2) all natural ingredients that are designed to boost the human immune system.

2. A delivery system for patented Formulation 100 (U.S. Pat. No. 6,667,063 B2)
The Emuno Bar will be the only Dark Chocolate covered functional food bar containing the new revolutionary patented Formulation 100.
